(19) United States
(12) Patent Application Publication
(10) Pub. No.: US 2009/0191254 A1
(43) Pub. Date: Jul. 30, 2009

(54) INHIBITION OF BIOFILM FORMATION USING BACTERIOPHAGE

(75) Inventors: John J. Curtin, Co. Limerick (IE); Rodney M. Donlan, Atlanta, GA (US)

Correspondence Address: KLARQUIST SPARKMAN, LLP, 121 S.W. SALMON STREET, SUITE 1600, PORTLAND, OR, 97204, US

(73) Assignee: The Govt. of the U.S.A., Centers for Disease Control and Prevention, Atlanta, GA (US)

(21) Appl. No.: 11/720,908

(22) PCT Filed: Dec. 6, 2005
(86) PCT No.: PCT/US2005/044498

§ 371 Date: Jun. 5, 2007

Related U.S. Application Data
(60) Provisional application No. 60/633,831, filed on Dec. 6, 2004.

(51) Int. Cl. A61F 2/04; A61K 35/76; A61K 9/10; A01N 63/00; B05D 3/00

(52) U.S. Cl. 424/423; 424/93.6; 424/484; 427/2.3

(57) ABSTRACT

Disclosed herein are indwelling medical devices suitable for introduction into the body of a subject, wherein the indwelling medical devices comprise one or more surfaces on which a bacterial biofilm can form, and an effective amount of a bacteriophage composition carried by one or more of the surfaces, wherein the bacteriophage composition inhibits formation of the bacterial biofilm on the indwelling medical device. Also disclosed herein are methods of inhibiting the formation of bacterial biofilms on indwelling medical devices.

### CROSS REFERENCE TO RELATED APPLICATION

This application claims priority of, and incorporates by reference, U.S. Provisional Patent Application No. 60/633,831, filed Dec. 6, 2004.

### FIELD OF THE DISCLOSURE

This disclosure relates to medical devices resistant to bacterial biofilm formation. It further relates to methods for inhibiting bacterial biofilm formation on the surface of a medical device, particularly to methods that include one or more bacteriophages.

### BACKGROUND

Medical devices, particularly implantable medical devices (for example, catheters) frequently are colonized by microorganisms. This problem is particularly prevalent with indwelling medical devices that are adapted to remain inserted or implanted within a subject's body for a relatively long-term, that is, from about 30 days to about 12 months or longer. Bacteria often colonize on and around the medical device and, upon attaching to surfaces of the device, proliferate and form aggregates within a complex matrix consisting of extracellular polymeric substances, typically polysaccharides and polypeptides. The mass of attached bacteria and the associated extracellular polymeric substances is commonly referred to as a biofilm or slime (Costerton et al., J. Bacteriol. 176:2137-42, 1994). Antibacterial agents have difficulty penetrating biofilms and killing and/or inhibiting the proliferation of the bacteria within the biofilm (Costerton et al., Science 284:1318-22, 1999). The colonization of the bacteria on and around the device and the synthesis of the biofilm barrier may eventually result in encrustation, occlusion and failure of the device. The biofilm itself also serves as a sanctuary for pathogens, particularly bacterial pathogens including gram positive bacteria (such a Staphylococcus species and Streptococcus species), and gram negative bacteria (such as Enterobacter species and Pseudomonas species).

Staphylococcus aureus are an example of a highly virulent human pathogen. Both S. aureus and coagulase-negative staphylococci (for example, S. epidermidis) have emerged as major nosocomial pathogens associated with biofilm formation on implanted medical devices (Arciola et al., New Microbiol. 22:337-41, 1999 and O'Gara and Humphreys, J. Med. Microbiol. 50:582-87, 2001). These organisms are among the normal carriage flora of human skin and mucous membranes, making them prevalent complications during and after invasive surgery or prolonged hospital stays. As bacteria carried on both healthy and sick people, staphylococci are considered opportunistic pathogens that invade subjects via open wounds and via implanted medical devices.

Biofilm infections associated with staphylococci and other bacterial pathogens are a significant cause of morbidity and mortality, particularly in settings such as hospitals, nursing homes and infirmaries (Arciola et al., New Microbiol. 22:337-41, 1999 and O'Gara and Humphreys, J. Med. Microbiol. 50:582-87, 2001). Subjects at risk include infants, the elderly, the immuno-compromised, the immuno-suppressed, and those with chronic conditions requiring frequent hospital stays. Subjects with intravascular and other implanted prosthetic devices are at even greater risk from bacterial infections because of the introduction of foreign bodies, which serve to damage tissue and/or act as a surface for the formation of biofilms. Such infections can have chronic, if not fatal, implications.

Coating implantable medical devices with antibacterial agents is a promising approach for the control and prevention of these foreign body related infections. However, the emergence of antibiotic-resistant bacteria, particularly antibiotic-resistant staphylococci, suggests that additional means will be necessary to prevent the formation of biofilms on implantable medical devices (Davies, J., Science 264:375-82, 1994). Thus, there is a need to find new antibacterial agents with properties that inhibit biofilm associated bacterial infections and improve the durability of indwelling medical devices, without requiring their removal and/or replacement. The ability to prolong the effective life of indwelling catheters and other medical devices would also reduce the risk of morbidity associated with medical procedures for replacing these devices. A reduction in the number of such procedures would also help reduce medical costs.

### SUMMARY OF THE DISCLOSURE

The present disclosure relates to indwelling medical devices resistant to bacterial biofilm formation, such as a staphylococcal biofilm. Such indwelling medical devices include one or more surfaces on which a bacterial biofilm can form, and an effective amount of a bacteriophage composition carried by one or more of the surfaces, such that the bacteriophage composition inhibits formation of the bacterial biofilm on the indwelling medical device.

This disclosure also provides methods for inhibiting formation of a bacterial biofilm on a surface of an indwelling medical device by contacting the surface of the indwelling medical device with an effective amount of a composition containing one or more bacteriophages that inhibit formation of the bacterial biofilm prior to formation of the biofilm.

The foregoing and other features and advantages will become more apparent from the following detailed description of several embodiments, which proceeds with reference to the accompanying figures.

### BRIEF DESCRIPTION OF THE FIGURES

FIG. 1 is a photograph of a modified drip flow biofilm reactor, overhead view. Components labeled in FIG. 1 include effluent tubes, reaction chambers (four), catheter segments, chamber lid, injection ports, and influent tubes.

FIG. 2 is a photograph of a modified drip flow biofilm reactor, side view, showing the removal legs.

FIG. 3 is a photograph of a modified drip flow biofilm reactor, overhead view. The chamber lid has been removed, and the catheter segments within the reactor chambers are shown.

FIG. 4 is a photograph of a modified drip flow biofilm reactor, attached via a pump to a batch culture vessel and a sterile media vessel; also attached to the reactor is a waste vessel.

FIG. 5 is a graph showing S. epidermidis 414 growth at 2, 6, and 24 hours on untreated catheters (diamonds), bacteriophage 456-treated catheters (squares), and bacteriophage 456-treated catheters supplemented with magnesium and calcium (open circles). Error bars represent±standard deviation (n=3).

FIG. 6 is a graph showing S. epidermidis 414 growth at 2, 6, and 24 hours on untreated catheters (diamonds), untreated catheters supplemented with magnesium and calcium (squares), heat inactivated bacteriophage 456-treated catheters (open circles), and heat inactivated bacteriophage 456-treated catheters supplemented with magnesium and calcium (open squares). Error bars represent±standard deviation (n=3).

FIG. 7 is a graph of viable bacteriophage 456 recovered at 2, 6, and 24 hours from the lumens of bacteriophage 456-treated catheters (squares), bacteriophage 456-treated catheters supplemented with magnesium and calcium (open circles), and bacteriophage 456-treated, serum coated catheters supplemented with magnesium and calcium (triangles). Error bars represent±standard deviation (n=3).

FIGS. 8A and 8B are scanning electron microscopy photomicrographs showing S. epidermidis 414 biofilm on an untreated catheter surface at 1000× magnification (FIG. 8A) and 3000× magnification (FIG. 8B).

FIGS. 9A and 9B are scanning electron microscopy photomicrographs showing S. epidermidis 414 biofilm on bacteriophage 456-treated catheter surface supplemented with divalent cations at 1000× magnification (FIG. 9A) and 3000' magnification (FIG. 9B).

FIG. 10 is a graph showing S. epidermidis 414 growth at 2, 6, and 24 hours on serum-coated untreated catheters (diamonds) and bacteriophage 456-treated, serum-coated catheters supplemented with divalent cations (open circles). Error bars represent±standard deviation (n=3).

### DETAILED DESCRIPTION

### I. Abbreviations

° C.: degrees Celsius

h: hour(s)

mDFR: modified drip flow biofilm reactor

MHA: Mueller Hinton agar

MHB: Mueller Hinton broth

min: minute(s)

ml: milliliter

rpm: revolutions per minute

μg: microgram(s)

μl: microliter(s)

μm: micrometer(s)

sec: second(s)

SEM: scanning electron microscopy

PFU: plaque forming units

CFU: colony forming units

### II. Terms

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and

other similar references.

In order to facilitate review of the various embodiments of this disclosure, the following explanations of specific terms are provided:

Animal: Living multi-cellular vertebrate organisms, a category that includes, for example, mammals and birds. The term mammal includes both human and non-human mammals. Similarly, the term “subject” includes both human and veterinary subjects, for example, humans, non-human primates, dogs, cats, horses, and cows.

Antibacterial enzyme: Any enzyme (such as a proteolytic, pore-forming, degradative or inhibitory enzyme) that kills or damages a bacterial species or particular strain thereof. The result may be achieved by damaging the cell wall of the bacteria, disrupting cell membranes associated with the cell wall or within the bacteria, inhibiting protein synthesis within the bacteria, disrupting the sugar backbone, or by any other mechanism attributed to a peptide or protein considered by those skilled in the art to be an antibacterial enzyme. The enzyme may be a natural, wild-type enzyme, modified by conventional techniques, conjugated to other molecules, recombinantly expressed, or synthetically constructed.

Examples of antibacterial enzymes include, but are not limited to, a lytic enzyme, an acylase, an aminopeptidase, an amylase, a carbohydrase, a carboxypeptidase, a catalase, a cellulase, a chitinase, a cutinase, a cyclodextrin glycosyltransferase, a deoxyribonuclease, an esterase, an alpha-galactosidase, a beta-galactosidase, a glucoamylase, an alpha-glucosidase, a beta-glucosidase, a haloperoxidase, an invertase, a laccase, a lipase, a mannosidase, an oxidase, a pectinolytic enzyme, a peptidoglutaminase, a peroxidase, a phytase, a polyphenoloxidase, a proteolytic enzyme, a ribonuclease, a transglutaminase, a xylanase, and lysostaphin.

Antibiotic: A chemical substance, produced by microorganisms, naturally occurring, or synthetically prepared, that has the capacity to inhibit the growth or replication of bacteria. Exemplary antibacterial antibiotics include, but are not limited to, a beta-lactam, a cephalosporin, an aminoglycoside, a sulfonamide, a macrolide, a tetracycline, a silver salt, and the like. Such antibiotics can be either bacteriostatic or bactericidal.

Bacteriophage: A. virus capable of infecting a bacterium. Certain bacteriophages are lytic, that is, they lyse the bacteria that they infect. The virus particle attaches to the bacterial cell wall and viral nucleoprotein enters the cell, resulting in the synthesis of virus and its liberation on physical disruption (lysis) of the cell. Bacteriophages are usually specific for bacterial species, but they may be strain-specific or may infect more than one species of bacteria. Exemplary bacteriophages include, but are not limited to, staphylococcal bacteriophages (such as bacteriophage 456) and bacteriophages that infect pseudomonal and enterococcal species. Lytic bacteriophages are particularly suitable for the methods disclosed herein.

Biofilm: A mass of microorganisms attached to a surface, such as a surface of a medical device, and the associated extracellular substances produced by one or more of the attached microorganisms. The extracellular substances are typically polymeric substances that commonly include a matrix of complex polysaccharides, proteinaceous substances and glycopeptides. The microorganisms may include, but are not limited to, bacteria, fungi and protozoa. In a “bacterial biofilm,” the microorganisms include one or more species of bacteria. The nature of a biofilm, such as its structure and composition, may depend on the particular species of bacteria present in the biofilm. Bacteria present in a biofilm are commonly genetically or phenotypically different than corresponding bacteria not in a biofilm, such as isolated bacteria or bacteria in a colony. “Polymicrobic biofilms” are biofilms that include a plurality of bacterial species.

Effective amount: The “effective amount” of a composition is the quantity of a composition sufficient to achieve a desired result. For instance, this can be the amount of a composition containing a sufficient dose of bacteriophage sufficient to inhibit the formation of a bacterial biofilm on a surface of an indwelling medical device. The effective amount of a composition will depend on, for example, the bacteriophage(s) contained in the composition, the concentration of the bacteriophage(s) in the composition, the amount of time the composition is in contact with the surface, the temperature at which the interaction between the composition and the surface takes place, and the like. In another example, an “effective amount” can refer to an amount of a substance sufficient to enhance the growth or stability of a bacteriophage, such as divalent metal cations.

Indwelling medical device: Any device for use in the body of a subject, such as intravascular catheters (for example, intravenous and intra-arterial), right heart flow-directed catheters, Hickman catheters, arteriovenous fistulae, catheters used in hemodialysis and peritoneal dialysis (for example, silastic, central venous, Tenckhoff, and Teflon catheters), vascular access ports, indwelling urinary catheters, urinary catheters, silicone catheters, ventricular catheters, synthetic vascular prostheses (for example, aortofemoral and femoropopliteal), prosthetic heart valves, prosthetic joints, orthopedic implants, penile implants, shunts (for example, Scribner, Torkildsen, central nervous system, portasystemic, ventricular, ventriculoperitoneal), intrauterine devices, tampons, dental implants, stents (for example, ureteral stents), artificial voice prostheses, tympanostomy tubes, gastric feeding tubes, endotracheal tubes, pacemakers, implantable defibrillators, tubing, cannulas, probes, blood monitoring devices, needles, and the like. A subcategory of indwelling medical devices are implantable devices that are typically more deeply and/or permanently introduced into the body. Indwelling medical devices can be introduced by any suitable means, for example, by percutaneous, intravascular, intraurethral, intraorbital, intratracheal, intraesophageal, stomal, or other route, or by surgical implantation, for example intraarticular placement of a prosthetic joint.

Inhibiting formation of a biofilm: Inhibiting the formation of a biofilm refers to avoiding the partial or full development or progression of a biofilm, for example, on a surface, such as a surface of an indwelling medical device.

Lumen: The cavity or channel within a tube or tubular object.

Releasably absorbed: A substance is releasably absorbed in a material if the substance is incorporated into the material such that substance is effectively released from the material under appropriate conditions. Releasably absorbed substances are to be distinguished from substances that are more permanently bonded or adhered to a material, such as substances covalently bonded to a material or substances incorporated into an erodible material. In particular examples, a releasably absorbed substance is one that is incorporated into the fluid phase of a viscous gel. The releasably absorbed substance can, for example, leech out from or with the fluid phase of the viscous gel, such as to the surface of the viscous gel. In particular examples, the releasably absorbed substance is stable in the material under certain conditions, and is released under other conditions. For example, an indwelling medical device may release a releasably absorbed substance when implanted into the body of a subject, while the substance is not released from the medical device during storage. The conditions that may result in the release of a releasably absorbed substance may include the temperature, pressure, osmotic potential, electrical potential, and carrier or medium surrounding the material.

Staphylococcal: Relating to or caused by any organism of the genus Staphylococcus. Exemplary organisms of the genus Staphylococcus include, but are not limited to, S. aureus and S. epidermidis.

Sterile: Free from living organisms and especially microorganisms such as bacteria, fungi, viruses, and protozoa.

Surface: The outer part or external aspect of an object that can interact with the environment of the object.

Surfactant: A substance which changes the nature of a surface, including water surface tension. Examples of surfactants include, but are not limited to, biosurfactants (such as glycolipids, lipopeptides, depsipeptides, phospholipids, substituted fatty acids, lipopolysaccharides, surlactin, surfactin, visconsin, and rhamnolipids), sodium dodecyl sulfate, quaternary ammonium compounds, alkyl pyridinium iodides, Tween 80, Tween, 85, Triton X-100, and the like.

Viscous gel: A colloid in which the solid disperse phase forms a network in combination with the fluid continuous phase to produce a high viscosity yet flowable or gelatinous material that is capable of adhering to a surface, such as a wall that forms the lumen of a catheter. Examples include, but are not limited to, gels formed by large molecules acting as the solid disperse phase, such as collagen, gelatin, agarose, acrylamide, and starch. A “hydrogel” is a colloid in which a solid disperse phase (for example, macromolecules such as collagen, gelatin, agarose, acrylamide, starch, or the like) forms a network in combination with the fluid continuous phase, and the fluid is water.

Viscous material: A high-viscosity material that is flowable, or gelatinous, that is capable of adhering to a surface, such as the surface of a medical device. A viscous gel is an example of a viscous material.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. The materials, methods, and examples are illustrative only and not intended to be limiting.

### III. Overview of Several Embodiments

Provided herein in various embodiments are indwelling medical devices that are useful for introduction into the body of a subject. In one embodiment the medical device comprises one or more surfaces on which a bacterial biofilm can form, and an effective amount of a bacteriophage composition carried by one or more of the surfaces, wherein the bacteriophage composition inhibits formation of the bacterial biofilm on the indwelling medical device. In a specific, non-limiting example, the medical device is suitable for surgical implantation within the body, such as an indwelling medical device. Such medical devices include, for example, a catheter (for example, a urinary catheter or an intravascular catheter), a stent, a shunt, an endotracheal tube, a gastric feeding tube, an artificial joint, an intrauterine device, an artificial voice prosthesis, a needleless connector for a central venous catheter, a tympanostomy tube, an artificial heart valve, or a pacemaker. In further examples, the medical device contacts a body surface, but is not an indwelling medical device. For example, the medical device may be a contact lens, a suture, a bandage, or a patch.

In another embodiment, the medical device includes a lumen. In a particular implementation, the medical device is a catheter and the bacteriophage composition is present in the lumen of the catheter.

In a further embodiment, the bacteriophage composition comprises a lytic or staphylococcal bacteriophage composition; a specific, non-limiting example includes bacteriophage 456. In yet a further embodiment, the staphylococcal bacteriophage composition comprises 10¹⁰ PFU/ml of bacteriophage 456.

In still another embodiment, the bacteriophage composition further comprises a surfactant, an antibacterial enzyme, an antibiotic, a growth or activity enhancing agent, or combinations thereof Specific, non-limiting examples of an antibiotic include, for example, antibacterial antibiotics such as a beta-lactam, a cephalosporin, an aminoglycoside, a sulfonamide, a macrolide, a tetracycline, a silver salt, or combinations thereof Examples of growth or activity enhancing agents include agents that help stabilize or multiply bacteriophages, such as divalent metal cations. In particular examples, the divalent metal cation is Ca²⁺ or Mg²⁺, such as from CaCl₂ or MgCl₂.

In a further embodiment, the medical device is made by contacting the bacteriophage composition and a carrier (such as a liquid or a viscous gel) with the one or more surfaces of the device for a sufficient period of time to adhere the effective amount of the bacteriophage to the device, prior to introducing the device into the body of a subject. In a more particular embodiment, the bacteriophage is releasably incorporated into the carrier, such as being absorbed by the carrier. In a particular implementation, the carrier is a hydrogel. In yet a further embodiment, the medical device is sterile except for the bacteriophage composition. Exemplary medical devices include, but are not limited to, polymeric medical devices.

In certain embodiments, the bacteriophage composition includes a plurality of bacteriophages and inhibits the formation of a polymicrobic biofilm. In further embodiments, the bacteriophage composition inhibits, or contains bacteriophages that inhibit, the growth of bacteria of the family Enterobacteriacae; bacteria of the genus Staphylococcus, Enterococcus, or Streptococcus; or Staphylococcus aureus, Staphylococcus epidermidis, coagulase-negative staphylococci, Pseudomonas aeruginosa, Klebsiella pneumoniae, Escherichia coli, Enterococcusfaecalis, Enterococcusfaecium, Providencia stuartii, Proteus mirabilis, Morganella morganii, Acinetobacter calcoaceticus, Enterobacter aerogenes, Streptococcus agalactiae, Streptococcus avium, Streptococcus bovis, Streptococcus durans, Streptococcus faecalis, Streptococcus pneumoniae, Streptococcus pyogenes, Streptococcus suis, Streptococcus viridans, Streptococcus salivarius, or other clinically relevant bacteria.

In further embodiments, the bacteriophage incorporating medical devices inhibit biofilm formation for a prolonged period of time, such as at least twenty-four hours. In particular implementations, the bacteriophage incorporating medical devices inhibit biofilm formation for at least about a week, such as at least about thirty days, such as at least about a year.

Also provided herein in various embodiments are methods that are useful for inhibiting formation of a bacterial biofilm on a surface of an indwelling medical device. In one embodiment, the method comprises contacting the surface of the medical device with an effective amount of a composition comprising one or more bacteriophages that inhibit formation of the bacterial biofilm prior to formation of the biofilm. In one specific, non-limiting example, the bacteriophage is a lytic bacteriophage. In another specific, non-limiting example, the bacteriophage inhibits formation of a specific target bacteria, such as staphylococci that are capable of forming a staphylococcal bacteria biofilm. Exemplary staphylococcal bacteria biofilms include, but are not limited to, S. aureus and S. epidermidis biofilms. In another embodiment, the method includes contacting the surface of the medical device with an effective amount of a composition that includes one or more staphylococcal bacteriophages, such as lytic staphylococcal bacteriophages, that inhibit formation of the bacterial biofilm prior to formation of the biofilm.

In a further embodiment, the bacteriophages are attached or adhered to the surface of the medical device with a viscous material, such as a gel (for example, a hydrogel), that contains the one or more bacteriophages. In particular implementations, the bacteriophages are releasably absorbed by the viscous material. In yet another embodiment, the viscous gel further contains a surfactant, an antibacterial enzyme, an antibiotic, a growth or activity enhancing agent, or combinations thereof. Specific, non-limiting examples of the antibiotic include, for example, a beta-lactam, a cephalosporin, an aminoglycoside, a sulfonamide, a macrolide, a tetracycline, a silver salt, or combinations thereof. In particular examples, growth or activity enhancing agent includes divalent metal cations, such as are Ca²⁺ or Mg²⁺.

In a yet further embodiment, the medical device includes a surface having a coating, such as a viscous gel or material capable of forming a viscous gel, capable of absorbing a bacteriophage composition, such as a bacteriophage solution. In particular implementations, the bacteriophage composition is releasably absorbed by the viscous gel. The medical device is contacted with the bacteriophage composition for a period of time sufficient for the surface of the medical device to absorb at least a portion of the bacteriophage composition. In a particular implementation, the medical device is coated with a hydrogel. In further implementations, the medical device is pre-coated with the absorbent coating, while in other implementations, the embodiment includes coating the medical device with a viscous gel (such as a hydrogel), or material capable of forming a viscous gel.

In a specific disclosed example, the medical device is suitable for surgical implantation within the body of a subject, and is introduced into the body of a subject. Such medical devices include indwelling medical devices, for example, a catheter, a stent, a shunt, an endotracheal tube, a gastric feeding tube, an artificial joint, an artificial heart valve, an intrauterine device, an artificial voice prosthesis, a tympanostomy tube, a needleless connector for a central venous catheter, or a pacemaker. Particularly useful examples are intravascular catheters (such as a central venous line) and intraurethral catheters (such as a bladder catheter), which are sometimes left in place for days or weeks.

In another specific example of the provided method, the medical device includes a lumen, which is often the site of bacterial biofilm occlusion and/or infection. In yet another specific example of the provided method, the medical device is a catheter and the bacteriophage composition is present in the lumen of the catheter. In a further specific example of the provided method, the bacteriophage composition is present in the lumen of the catheter prior to insertion of the catheter into a subject. Alternatively, the bacteriophage composition is present in the lumen of the catheter only after insertion of the catheter into a subject.

In particular embodiments, the bacteriophage incorporating medical device is effective at preventing biofilm formation for an extended, or prolonged, period of time, such as at least about 24 hours, such as a least about a week. In particular examples, the medical device is effective at preventing biofilm formation for at least about a month. In particular examples, the medical device is effective at preventing biofilm formation for at least about 6 months, such as at least about a year.

In further embodiments, the bacteriophage incorporating medical device is capable of preventing biofilm formation after being inserted into the body of a subject and coated with substances in the body of the subject. In particular examples, the substances that coat the medical device are platelets, plasma, or host proteins such as albumin, fibrinogen, fibronectin, and laminin.

In another aspect, the present disclosure provides methods of forming a medical device. A surface of the medical device is coated with a viscous material, such as a viscous gel., One or more bacteriophages are incorporated into the viscous material. In particular implementations, the bacteriophage is releasably absorbed by the viscous material. In particular examples, the bacteriophage is incorporated into the viscous material before coating the surface of the medical device. In further examples, the bacteriophage is incorporated into the viscous material after the surface of the medical device is coated with the viscous material. In a particular example, a bacteriophage solution is contacted with a viscous material-coated surface of the medical device.

In a further embodiment, the present disclosure provides methods for introducing an indwelling medical device into the body of a subject. A medical device coated with a viscous material that includes a bacteriophage is provided. The medical device is then introduced into the body of the subject. In particular implementations, the viscous material is a viscous gel, such as a hydrogel. In particular examples, the bacteriophage is releasably absorbed by the viscous material. In further implementations, the medical device remains in the body of the subject for a prolonged period of time, such as at least twenty-four hours, such as at least a week. In particular examples, the medical device remains in the body of the subject for at least thirty days, such as at least a year.

### IV. Indwelling Medical Devices Including One or More Bacteriophage-Treated Surfaces for the Inhibition of Biofilm Formation

Bacterial biofilm formation on the surfaces of implantable or insertable medical devices adapted for long-term implantation, for example, from about 30 days to 12 months or longer, can result in eventual encrustation and failure of the device. Additionally, the proliferation of bacteria within the biofilm can lead to localized infections as well as difficult to treat, and sometimes fatal, systemic infections, such as bacteremia and bacterial sepsis. The extracellular substances that comprise the biofilm matrix can act as a barrier that protects and isolates the bacteria resident within the biofilm from normal immunological defense mechanisms, such as antibodies and phagocytes, as well as from antimicrobial agents including surfactants, antibacterial enzymes and antibiotics. The biofilm also facilitates the growth and proliferation of bacteria resident within the biofilm.

The present disclosure provides indwelling medical devices adapted for use (including long term use and permanent or semi-permanent implantation) with substantially reduced risk of biofilm accumulation on the surfaces of the medical devices, and the resultant likelihood of premature failure of the devices due to encrustation and occlusion by such biofilm. In some embodiments of the present disclosure, the medical device is intended to remain indwelling for a relatively long period of time, such as from about 30 days to about 12 months, or longer. However, it is understood that the device may be indwelling for a shorter period of time, such as 30 days or less, as well. For example, in certain embodiments, a prolonged period of time may be a period longer than 24 hours, such as a week.

A bacterial biofilm may include an integrated community of two or more bacteria (polymicrobic biofilms), or predominantly a specific bacterium (Palmer and White, Trends in Microbiology 5:435-40, 1997 and Costerton et al., Annual Reviews of Microbiology 41:435-64, 1987). In the present disclosure, the one or more bacteria may be any bacteria involved in biofilm formation, including both aerobic bacteria and anaerobic bacteria or Gram positive and Gram negative bacteria. Specific, non-limiting examples include Staphylococcus species, such as Staphylococcus aureus, and Pseudomonas species, such as Pseudomonas aeruginosa, bacilli, and enterococci.

The bacteriophage compositions disclosed herein can include staphylococcal bacteriophage. Exemplary staphylococcal bacteriophage (including their respective host(s)) are shown in Table 1, below (from Dr. Tyrone Pitt, Health Protection Agency (HPA), London, England, unless otherwise indicated). The bacteriophage compositions disclosed herein also include pseudomonal bacteriophage.

TABLE 1
Staphylococcal Bacteriophage

| Phage | Host |
|---|---|
| 15 | S. epidermidis 414 |
| 37 | S. simulans |
| 155 | S. cohnii |
| A6C | S. epidermidis A6C |
| 48 | S. epidermidis 414, S. epidermidis 48 |
| 82 | S. epidermidis 414 |
| 456 | S. epidermidis 414 |
| 157A | S. epidermidis 157A |
| 471A | S. epidermidis 414 |
| 459 | S. epidermidis 414, S. epidermidis 459 |
| 392 | S. epidermidis 414 (not part of HPA set) |

In particular embodiments, the bacteriophage composition inhibits, or contains bacteriophages that inhibit, the growth of bacteria of the family Enterobacteriacae; bacteria of the genus Staphylococcus, Enterococcus, or Streptococcus; or Staphylococcus aureus, Staphylococcus epidermidis, coagulase-negative staphylococci, Pseudomonas aeruginosa, Klebsiella pneumoniae, Escherichia coli, Enterococcus faecalis, Enterococcus faecium, Providencia stuartii, Proteus mirabilis, Morganella morganii, Acinetobacter calcoaceticus, Enterobacter aerogenes, Streptococcus agalactiae, Streptococcus avium, Streptococcus bovis, Streptococcus durans, Streptococcus faecalis, Streptococcus pneumoniae, Streptococcus pyogenes, Streptococcus suis, Streptococcus viridans, Streptococcus salivarius, or other clinically relevant bacteria.

In one embodiment, surfaces of an indwelling medical device prone to bacterial biofilm formation can be subjected to the methods of the present disclosure as a preventative measure prior to any biofilm formation to substantially avoid biofilm formation. Alternatively, at the first indication of biofilm formation, the methods may be used to prevent further biofilm formation and to remove the biofilm that has become deposited on a surface. Furthermore, in situations where there is a heavy build-up of biofilm on a surface, the methods may be used to reduce the level of biofilm or to remove it partially or completely.

The indwelling medical devices which are amenable to treatment with the disclosed bacteriophage compositions generally have surfaces composed of thermoplastic or polymeric materials such as polyethylene, Dacron, nylon, polyesters, polytetrafluoroethylene, polyurethane, latex, silicone elastomers, and the like. The surfaces of the device may be smooth or rough, for example, a smooth polymeric surface of a catheter lumen or a relatively rough Dacron patch for repairing an abdominal or vascular defect. Indwelling medical devices with metallic surfaces are also amenable to treatment with the disclosed bacteriophage compositions. Such devices, for example bone and joint prosthesis, can be coated by a cement mixture including the disclosed bacteriophage compositions. During implant use, the bacteriophages leach from the cement into the surrounding prosthesis surface environment. Particular indwelling medical devices especially suited for application of the bacteriophage compositions of this disclosure include intravascular, peritoneal, pleural and urological catheters, heart valves, gastric feeding tubes, endotracheal tubes, tympanostomy tubes, intrauterine devices, artificial voice prostheses, prosthetic joints, stents, needleless connectors for central venous catheters, cardiac pacemakers, vascular shunts, and orthopedic, intraocular, or penile prosthesis.

Various methods can be employed to treat the surfaces of an indwelling medical device with the bacteriophage compositions. For example, one of the simplest methods is to flush the surfaces of the medical device with a solution of the bacteriophage composition. Generally, treating the surfaces by a simple flushing technique would require convenient access to the medical device. For example, catheters, are generally amenable to flushing with a solution containing the bacteriophage compositions disclosed herein. For use in flushing solutions, high titer (for example, 10¹⁰ PFU/ml or higher) bacteriophage stocks are used. The flushing solution would normally be composed of sterile media or sterile normal saline solutions. The bacteriophage composition may also be painted or sprayed on the medical device. In particular implementations, the medical device is dipped or immersed in the composition.

Another method of treating the surfaces of an ihdwelling medical device with the bacteriophage compositions is to first apply or adsorb to the surfaces of the medical device a viscous gel layer (such as a hydrogel) followed by addition (via flushing or direct instillation of high titer stock) of the bacteriophage composition. Alternatively, the bacteriophages may be present in the gel when it is applied to the device. In another implementation, the device is pre-coated with the viscous layer. Exemplary components of the viscous gel layer include, but are not limited to, polylactic acid, polyglycolic acid and copolymers and mixtures thereof such as poly(L-lactide) (PLLA), poly(D,L-lactide) (PLA); polyglycolic acid [polyglycolide (PGA)], poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-cotrimethylene carbonate) (PGA/PTMC), poly(D,L-lactide-co-caprolactone) (PLA/PCL), poly(glycolide-co-caprolactone) (PGA/PCL); polyethylene oxide (PEO), polydioxanone (PDS), polypropylene fumarate, poly(ethyl glutamate-co-glutamic acid), poly(tert-butyloxy-carbonylmethyl glutamate), polycaprolactone (PCL), polycaprolactone co-butylacrylate, polyhydroxybutyrate (PHBT) and copolymers of polyhydroxybutyrate, poly(phosphazene), poly(phosphate ester), poly(amino acid) and poly(hydroxy butyrate), polydepsipeptides, maleic anhydride copolymers, polyphosphazenes, polyiminocarbonates, poly[(97.5% dimethyl-trimethylene carbonate)-co-(2.5% trimethylene carbonate)], cyanoacrylate, polyethylene oxide, hydroxypropylmethylcellulose, polysaccharides such as hyaluronic acid, chitosan and regenerate cellulose, and proteins such as gelatin and collagen, and mixtures and copolymers thereof, among others.

Furthermore, the bacteriophage compositions can be coadministered, simultaneously or alternately, with other agents, such as antibacterial agents, so as to more effectively inhibit bacterial biofilm formation, or, alternatively, prevent further biofilm formation and to remove the biofilm that has become deposited on a surface. For example, the bacteriophage compositions can include a surfactant, an antibacterial enzyme, an antibiotic, a growth or activity enhancing agent, or combinations thereof.

As disclosed herein, exemplary surfactants include biosurfactants (such as glycolipids, lipopeptides, depsipeptides, phospholipids, substituted fatty acids, lipopolysaccharides, surlactin, surfactin, visconsin, and rhamnolipids), sodium dodecyl sulfate, quaternary ammonium compounds, alkyl pyridinium iodides, Tween 80, Tween, 85, Triton X-100, and the like. Exemplary antibacterial enzymes are a lytic enzyme, an acylase, an aminopeptidase, an amylase, a carbohydrase, a carboxypeptidase, a catalase, a cellulase, a chitinase, a cutinase, a cyclodextrin glycosyltransferase, a deoxyribonuclease, an esterase, an alpha-galactosidase, a beta-galactosidase, a glucoamylase, an alpha-glucosidase, a beta-glucosidase, a haloperoxidase, an invertase, a laccase, a lipase, a mannosidase, an oxidase, a pectinolytic enzyme, a peptidoglutaminase, a peroxidase, a phytase, a polyphenoloxidase, a proteolytic enzyme, a ribonuclease, a transglutaminase, a xylanase, and lysostaphin. Examples of antibiotics include antibiotics that interfere with or inhibit cell wall synthesis, such as penicillin, nafcillin, oxacillin, and other beta-lactam antibiotics; cephalosporins such as cephalothin; glycopeptides such as vancomycin; and other polypeptides. In particular examples, the growth or activity enhancing agent is a divalent metal cation, such as Ca²⁺ or Mg²⁺.

Suitable coatings for indwelling medical devices include various hydrogel coatings. One or more bacteriophages can be incorporated into the hydrogel before or after the hydrogel is applied to the medical device. Suitable hydrogels include organic coatings formed by applying a mixture of an isocyanate, a polyol, polyvinylpyrrolidone, and a carrier to the surface of a medical device, as discussed in U.S. Pat. No. 5,160,790, columns 4 through 13 of which are expressly incorporated by reference herein. Silane copolymers can also be used to form suitably coated medical devices. For example, columns 4 through 12 of U.S. Pat. No. 6,908,681, which are expressly incorporated by reference herein, disclose silane copolymers formed by reacting one or more polyisocyanates, a silane, and molecules having at least two functional groups that are reactive with isocyanate. Columns 4 through 10 and 14 through 21 of U.S. Pat. No. 6,596,402, which are expressly incorporated by reference herein, discuss medical device coatings formed by contacting the medical device with a copolymer including an organic group that reacts with water to form a silanol group. Suitable hydrogel coated catheters include LubriSil catheters, available from C. R. Bard, Inc., of Covington, Ga.

In particular implementations, the hydrogel coating of the medical device includes an antimicrobial agent or another antibiotic, such as silver. Suitable catheters having a hydrogel coating that includes an antimicrobial agent include the Bardex catheters, available from C.R. Bard, Inc., that incorporate the Bacti-Guard silver antimicrobial agent. Coatings that include a fast-acting antimicrobial agent and a long-lasting antimicrobial agent are disclosed in U.S. Pat. No. 6,579,539, columns 2 through 6 of which are expressly incorporated by reference herein.

The subject matter of the present disclosure is further illustrated by the following non-limiting Examples.

### EXAMPLES

### Example 1

### In Vitro System to Stimulate Biofilm Growth on a Surface of an Indwelling Medical Device

This example illustrates a system for S. epidermidis biofilm grown on a 15 surface of an indwelling medical device, using the lumen of a catheter as a model.

### Organisms and Culture Conditions

S. epidermidis 414 (HER 1292—Félix d'Hérelle Reference Center for Bacterial Viruses) was selected for this study. The organism stock was maintained at −80° C. Coagulase-negative staphylococcus phage 456 (Dean et al., J. Hyg. 71:261-270, 1973) (obtained from Health Protection Agency, Colindale, UK) was also used in this study and was maintained as a lyophilized preparation stored at 4° C. The phage was propagated using the soft agar overlay technique (Adams, M., Bacteriophages, Interscience Publishers, London, 1959; Gratia, A., Compt. Rend. Soc. Biol. 122:812, 1936) and crude high titer phage broth cultures were prepared as described by Adams (Adams, M., Bacteriophages, Interscience Publishers, London, 1959) using S. epidermidis 414 as the host strain in Mueller-Hinton Broth (MHB), (Difco, Becton Dickinson, Sparks, Md.) containing 3 mM MgCl₂ and 4 mM CaCl₂ (added as MgCl₂·6H₂O and CaCl₂·2H₂O). One ml of an 18 h culture of S. epidermidis 414 in MHB (cultured at 37° C.) was added to 49 ml of MHB containing 3 mM MgCl₂ and 4 mM CaCl₂. This was incubated at 37° C. with shaking at 250 revolutions per minute and the OD600 monitored until an absorbance of 0.3 was reached (Spectronic 21D Spectrophotometer, Spectronic Instruments Inc., Rochester, N.Y.). Phage 456 was added to a final concentration of 10⁶-10⁷ plaque forming units/ml (PFU/ml). The culture was allowed to stand for 15 min at 37° C. and then incubated for 18 h at 37° C. with shaking at 100 rpm. Host cell debris was pelleted by centrifugation (4000×g for 20 min) and the supernatant. containing phage filter sterilized (Millipore, Billerica, Mass.; 0.22 m pore size). The crude phage lysate was titered by plaque assay using the soft agar overlay technique on Mueller-Hinton agar (MHA) (Adams, M., Bacteriophages, Interscience Publishers, London, 1959; Gratia, A., Compt. Rend. Soc. Biol. 122:812, 1936), stored at 4° C., and used within a week.

### Catheters

Lubri-sil all-silicone 16 French Foley catheters were used (C. R. Bard, Covington, Ga.). These catheters are coated with a neutral hydrogel on both the inner and outer surfaces. Hydrogel-coated catheters were used in order to adsorb and help maintain the phage culture in the catheter lumen long enough for their effect on viable S. epidermidis cells to be determined.

### In Vitro Model of Biofilm Development on Hydrogel-Coated Catheters

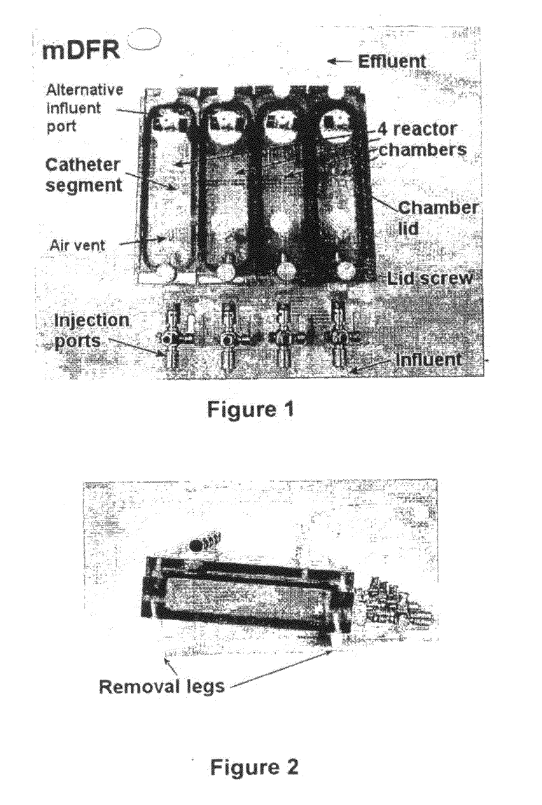

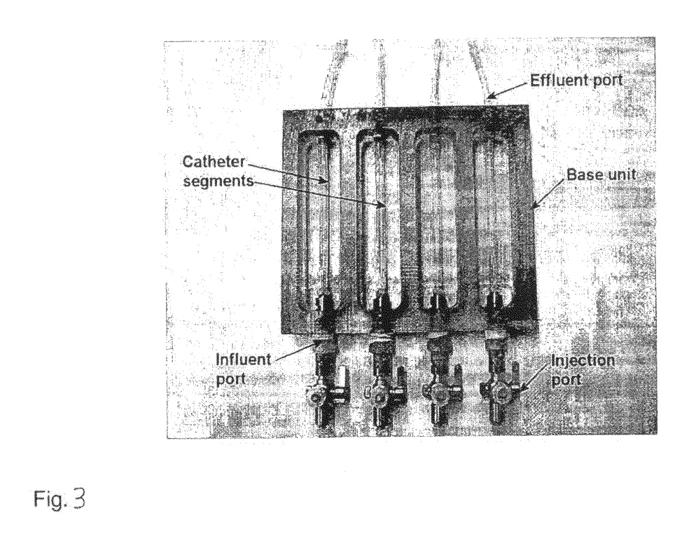

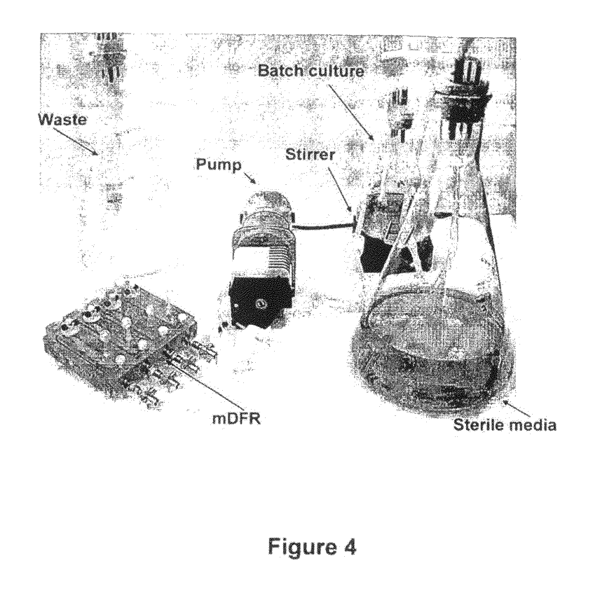

Biofilms were grown using a modified drip flow biofilm reactor (mDFR). A drip flow biofilm reactor (Biosurface Technologies, Bozeman, Mont.) was modified to provide a constant flow, rather than a drip flow, through catheters of various sizes and types, including central venous catheters and urinary catheters. An overhead view of the mDFR is shown in FIG. 1. The mDFR allows four catheter segments to be sealed in separate sterile chambers. Each chamber has a sealing lid. Injection ports can be used to add or remove liquids from the catheter segments. The mDFR can be raised to various angles on removal legs (FIG. 2). The original device was modified to allow the connection of catheter segments of any lumen size to influent and effluent ports within the device (FIG. 3). With reference to FIG. 4, the mDFR is assembled into a system that allows for bacterial batch culture to be pumped through the mDFR and the catheter segments inside, followed by sterile media to allow the biofilm to become established. The system includes a magnetic stirrer, a batch culture container, a sterile media reservoir, a peristaltic pump, a mDFR, and a waste reservoir.

Before each experiment, the device containing the catheters was sterilized using ethylene oxide gas. The mDFR was coupled to a batch culture of S. epidermidis in MHB and a sterile media reservoir containing half-strength MHB (FIG. 4). The culture (mid-exponential phase) was pumped through the mDFR for 2 h (1 ml/min), irrigating the catheter segments attached inside. The mean Colony Forming Units per ml (CFU/ml) of the batch culture ranged from 10⁸ to 10⁹ during this 2 h period. This was followed by irrigation for 22 h with sterile half-strength MHB (0.5 ml/min) to establish a biofilm. All mDFR experiments were carried out at 37° C. in triplicate.

### Example 2

### Inhibition of S. epidermidis Biofilm Growth Using Bacteriophage 456

### Phage Pre-Treatment of Catheter Surfaces

For the phage pre-treatment experiments, a crude MHB culture of phage 456 with a titer between 1×10¹⁰ to 2.2×10¹⁰ PFU/ml was used. Prior to biofilm formation, each catheter segment in the mDFR was filled with the phage culture. The phage culture was incubated at 37° C. for 1 h within the catheter lumens before removal. Experiments were also conducted using MHB containing 3 mM MgCl₂ and 4 mM CaCl₂. Many phages, including staphylococcal phages, require divalent cations for efficient growth and multiplication (Kay, D., Br. J. Exp. Pathol. 33:228-235, 1952; Rountree, P. M., J. Gen. Microbiol. 12:275-287, 1955); thus divalent cations were added to the media to assess their effect on phage activity. Biofilms were grown using the supplemented media with and without phage pre-treatment of the catheter surface. Heat inactivated phages (80° C. for 3 h) were used as a control pre-treatment using both divalent cation supplement and non-supplemented MHB for biofilm growth. Biofilm formation took place as previously described over 24 h.

### Enumeration of Viable Adherent Organisms

During the course of experiments, single catheter segments were removed aseptically from the device and the fluid contained within the lumen collected at 2, 6, and 24 h. The catheter was cut into smaller sections each with an internal curved surface area of 1 cm². Three of these sections were sliced vertically into two halves and each halved section washed gently in 5 ml phosphate buffered saline (PBS, 7.2 pH) to remove planktonic and loosely adherent cells. Individual sections were subjected to high speed vortexing in 5 ml PBS for 15 s, followed by sonication for 10 min at 42 kHz (Branson 2510, Danbury, CT), further vortexting for 15 s, sonication for 5 min, and a final vortex for 15 s. Earlier studies indicated that the process removed essentially all of the viable S. epidermidis 414 cells from the surface of the catheter and that sonication was not associated with loss of viability of the cells in suspension (data not shown). In each experiment, the viable bacterial counts for the three 1 cm² sections of catheter were established and the mean viable count, expressed as CFU/cm², determined. In addition to determining viable bacterial counts, intra-luminal fluid from the catheter was titered for phage 456 using the soft agar overlay method (Adams, M., Bacteriophages, Interscience Publishers, London, 1959; Gratia, A., Compt. Rend. Soc. Biol. 122:812, 1936) where appropriate. The tubes containing the PBS and catheter sections were kept on ice as much as possible throughout the procedure to prevent further phage action on viable cells.

All experiments were performed in triplicate. Organisms recovered from the biofilms were subcultured on MHA and preserved at −80° C. The organisms were confirmed as S. epidermidis using a Vitek GPI card (biomerieux, Durham, N.C.). S. epidermidis cells that were still viable after exposure to phage 456 were tested to determine if their survival in the presence of the phages was due to acquired resistance. This was determined by plaque assay (Adams, M., Bacteriophages, Interscience Publishers, London, 1959; Gratia, A., Compt. Rend. Soc. Biol. 122:812, 1936) using a high titer phage 456 culture and comparing the titers and plaque assays of the recovered organisms with S. epidermidis 414 freshly revived from −80° C. stocks.

Analysis of Phage Effect on S. epidermidis Viability During Sampling Procedure

To determine if phage 456 was affecting the viability of S. epidermidis 414 cells on the catheter surface rather than during the biofilm sampling procedure, a suspension of S. epidermidis 414 was prepared in 5 ml PBS at 4° C. to the turbidity of a 0.5 MacFarland standard. A 1 ml aliquot was removed for viable cell count and 1 ml of a 10¹⁰ PFU/ml phage 456 culture in MHB was added to the remaining 4 ml suspension. The tube was subjected to the same biofilm removal procedure as used during mDFR experiments i.e., sonication and vortexing, followed by chilling on ice for 2 h (twice the length of time required for sampling during biofilm/phage experiments). The number of viable cells was then determined by plate count. In addition, phage pre-treated catheter segments from which biofilms had been recovered were stored at 4° C. for 24 h and the number of viable bacterial cells and phage determined again. All experiments were performed in triplicate.

Developing Biofilms on Hydrogel Catheters Pre-Treated with Phages and Human Serum

A conditioning film was simulated on the catheter lumen to assess its affect on biofilm formation and phage 456 effectiveness. Filter-sterilized whole human serum (complement inactivated at 56° C. for 30 min) was instilled into both phage pre-treated and untreated control catheter segment lumens in the mDFR and incubated for 2 h at 37° C. The presence of the serum conditioning film was confirmed by fluorescent microscopy. Catheter segments incubated with serum were cut in segments as before. The segments were washed with sterile PBS three times (1 min per wash), incubated with 3% bovine serum albumin at 37° C. for 1 h to prevent non-specific binding, and rewashed three times in PBS. The segments were then incubated with goat anti-human lgG (H+L) fluorescein isothiocyanate (FITC) conjugate (Zymed Laboratories, San Francisco, Calif.) for 1 h at room temperature and, washed three times in sterile PBS. Catheter segments not coated with serum were used as controls. All catheter segments were sliced lengthwise into 1 mm sections and examined using a Zeiss Axioplan 2 Imaging Fluorescent Microscope (10×, 20× and 100× objective lenses), (Carl Zeiss Light Microscopy, Göttingen, Germany) with a FITC filter (filter set #41001, excitation filter, 480/40×; emission filter, 535/50 m; dichroic mirror, 505 nm; Chroma Technology Corp., Rockingham, Vt.). Samples were photographed using a Zeiss Axiocam high resolution digital camera (Carl Zeiss Light Microscopy, Göttingen, Germany). Images were analyzed using Axiovision 4.0 software (Carl Zeiss Vision, München Hallbergmoos, Germany). Both test catheter segments and control segments were photographed using the same exposure time. Biofilms were developed on serum coated catheters either with or without phage pre-treatment as previously described (with divalent cation supplementation).

### Scanning Electron Microscopy (SEM)

The biofilm was visualized by SEM. Following the washing step, the biofilm was fixed with 5% glutaraldehyde (Ted Pella Inc., Redding, Calif.) in cacodylate buffer (0.67 M, pH 6.2) for 1 h and dehydrated through a series of aqueous ethanol washes (30 to 100%) for durations of 10 to 15 min. Specimens were treated with 1 drop of hexamethyldisilazane (Polysciences Inc., Warrington, Pa.), mounted on aluminum stubs with quick-drying silver paint (Ted Pella Inc., Redding, Calif.), coated in gold (Polaron SC7640 Sputter Coater, Thermo V G Scientific, UK), and viewed and photographed with a scanning electron microscope (Philips XL30 ESEM, FEI Co., Hillsboro, Oreg.). The entire surfaces of 1 cm² sections of catheter were examined and images were chosen that represented the typical field of view.

### Statistical Analysis

Bacterial and phage count data were log-transformed and differences in microbial recovery analyzed using the two-tailed P test and Excel 2003 (Microsoft Corporation, Redmond, Wash.). P values less than 0.05 were considered significant.

### Results

### In Vitro Model of Biofilm Development on Catheters After Phage Pre-Treatment

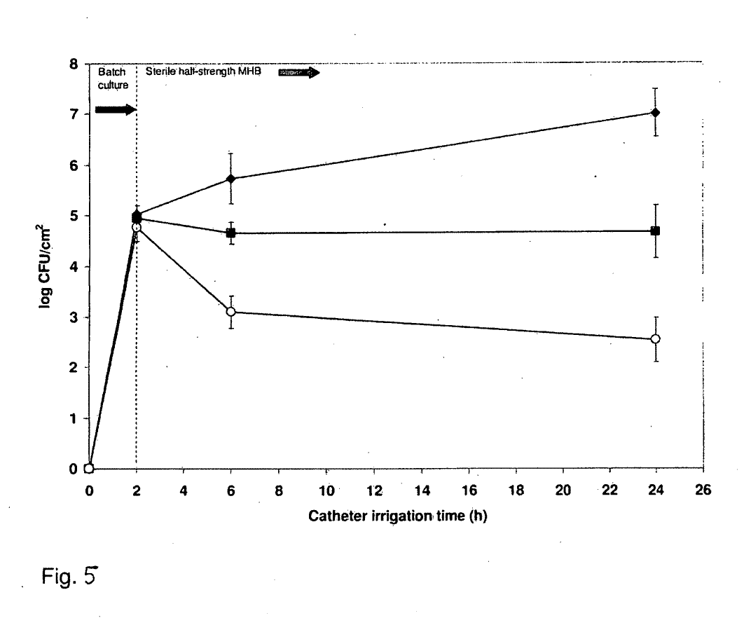

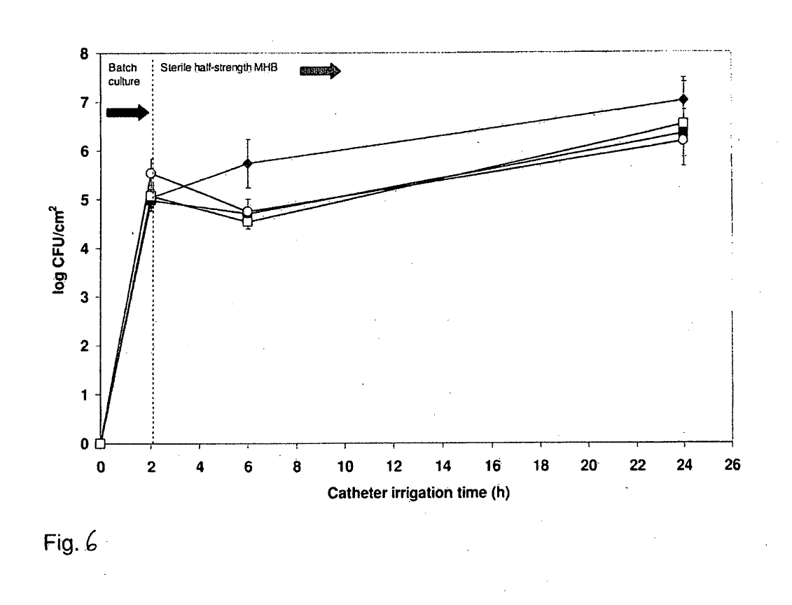

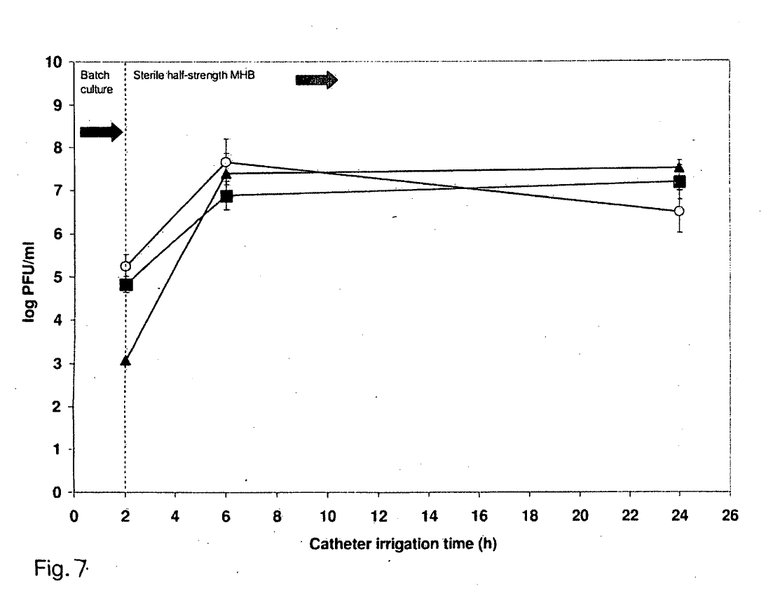

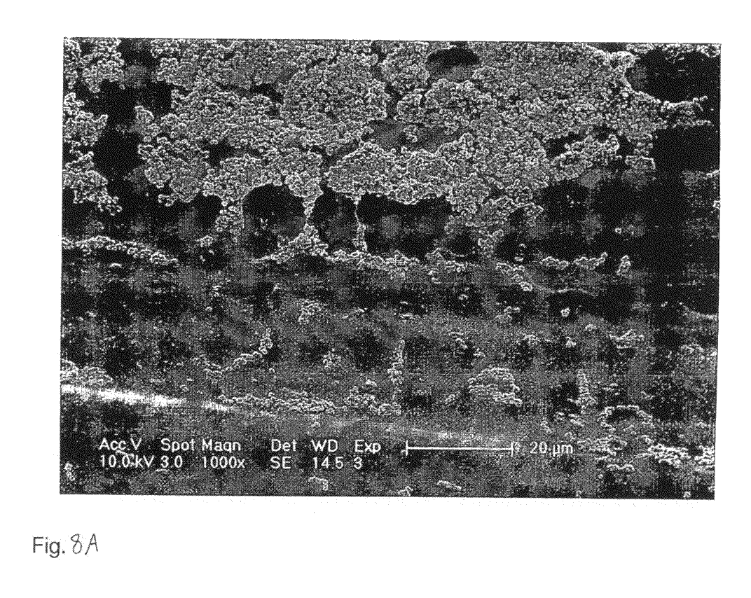

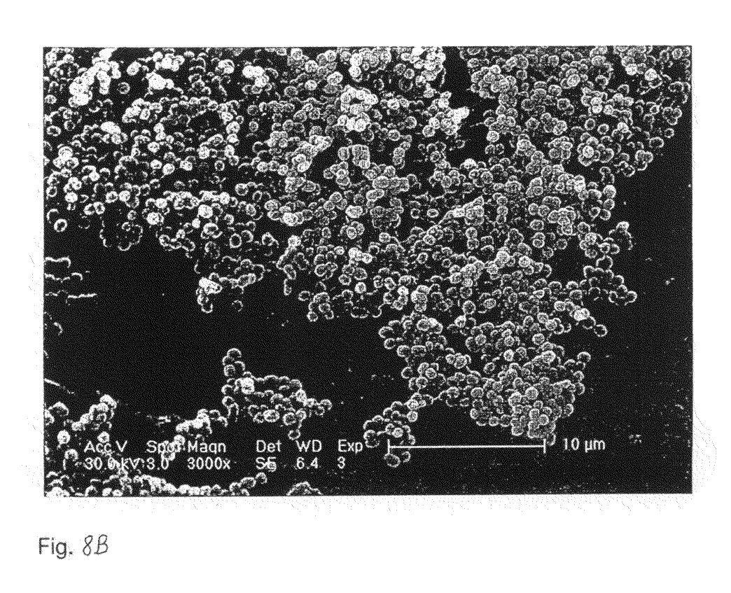

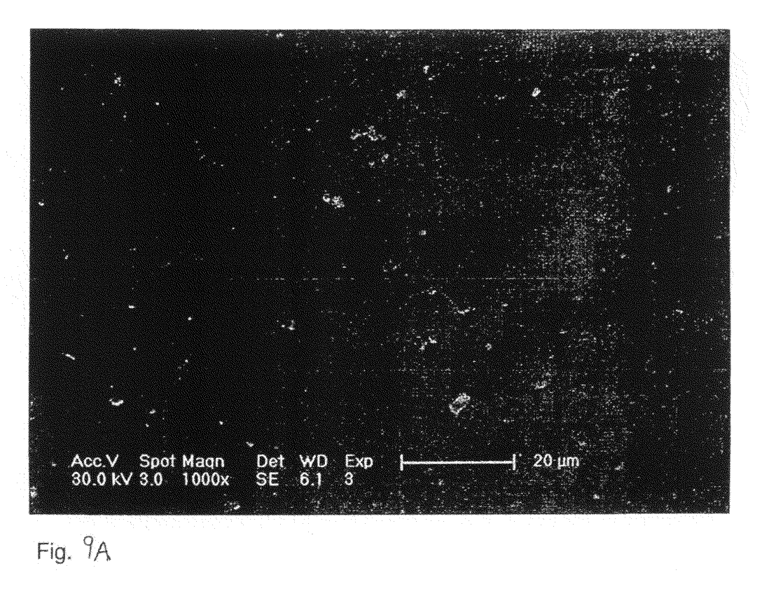

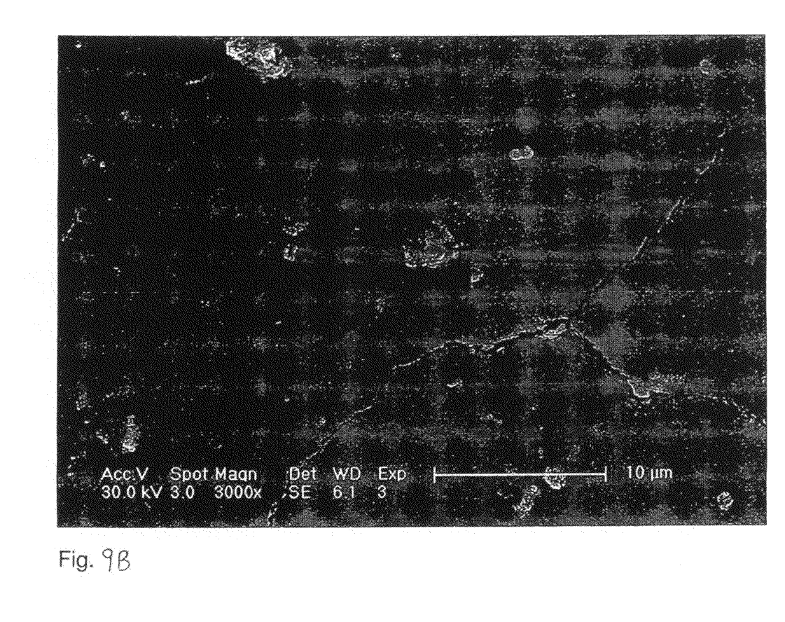

The mean viable count of S. epidermidis after 24 h biofilm formation was 7.01±0.47 log CFU/cm² of catheter. Pre-treatment of catheters with phage 456, with and without supplemental divalent cations, resulted in significant log reductions of 4.47 (p<0.0001) and 2.34 (p=0.001) in staphylococcal CFU/cm² respectively (FIG. 5). These represent greater than 99% reductions in viable cell numbers in both cases. Divalent cation supplementation of the growth medium without phage pre-treatment resulted in a non-significant reduction in bacterial viability (log 0.67 CFU/cm², p=0.053), as did pre-treatment with heat inactivated phage both with (log 0.48 CFU/cm², p=0.149) and without divalent cations (log 0.83 CFU/cm², p=0.065) (FIG. 6). The presence of phage 456 in the intra-luminal media was confirmed at all time points throughout the phage pre-treatment and subsequent biofilm growth experiments (FIG. 7). The presence of a biofilm on catheter surfaces was confirmed by SEM (FIGS. 8A and 8B). After phage 456 pre-treatment using supplemental divalent cations, the number of visibly attached S. epidermidis cells was considerably less in all fields of view (FIGS. 9A and 9B). Recovery of viable organisms from the catheter surfaces was not associated with acquired resistance of S. epidermidis 414 to phage 456 as determined by plaque assay (data not shown).

Effect of Phages on S. epidermidis Viability During Sampling Procedure

Catheter segments, sampled for viable S. epidermidis cells after pre-treatment of the catheter surface with phages and with supplemental divalent cations added to the media, were re-sampled after storage at 4° C. for 24 h. At the 2 h sampling point, a mean increase of log 0.02 CFU/ml (p=0.943) was recorded, while a reduction of log 0.28 CFU/ml (p=0.593) was observed at 6 h and a further reduction of log 0.01 CFU/ml (p=0.968) at 24 h. These data demonstrated that there was no significant effect on S. epidermidis viability by phage 456 in PBS at 4° C. The standardized 4 ml PBS suspension of S. epidermidis 414 inoculated with 1 ml of a 10¹⁰ PFU/ml culture of phage 456, and then subjected to the same biofilm removal and sampling procedure as described previously, did not result in a decrease in viable cell numbers as a result of phage action. An increase in mean viable S. epidermidis cell numbers of log 0.04 CFU/ml was recorded which was not statistically significant (p=0.586). These data indicate that there was no additional phage lysis of removed biofilm cells during the processing of catheter segments.

### Discussion

The results indicate that phages can be used to reduce growth of S. epidermidis biofilms. Biofilm formation on both hydrogel-coated and serum/hydrogel-coated silicone catheters was significantly reduced over 24 h in the presence of phage 456. Not only did the phage reduce the number of viable cells recovered but also reduced the number of cells adhering to the catheter surface as determined by SEM (FIGS. 8A, 8B, 9A, and 9B). The presence of divalent cations in the growth medium (MgCl₂ and CaCl₂) appeared to increase the effectiveness of phage 456 at reducing biofilm formation. In this example, the levels of divalent cations required for optimal phage activity (3 mM MgCl₂ and 4 mM CaCl₂) were low and many intravenous fluids (IV) include similar levels of magnesium and calcium. The addition of divalent cations to the media without phage pre-treatment of the catheter surface did not result in a significant increase or decrease in viable biofilm cells. While many of the S. epidermidis cells entering the catheter lumen may be infected by the phage before any substantial adherence to the surface takes place, it is likely that phage infection also occurs after bacterial cells have attached to the surface. Attachment of coagulase-negative staphylococci to catheter materials has been shown to occur rapidly, with adherent microorganisms visible on the surface by electron microscopy within minutes of exposure to the bacterial culture. No demonstrable effect of the phage on S. epidermidis adherence to the catheter material at 2 h was recorded. In fact, there was no significant difference between the number of cells recovered at 2 h from the non-phage pre-treated controls and the phage 456 pre-treated catheters (in the presence of divalent cations). This may account for the time needed for the phage to successfully infect and begin to lyse the S. epidermidis cells.

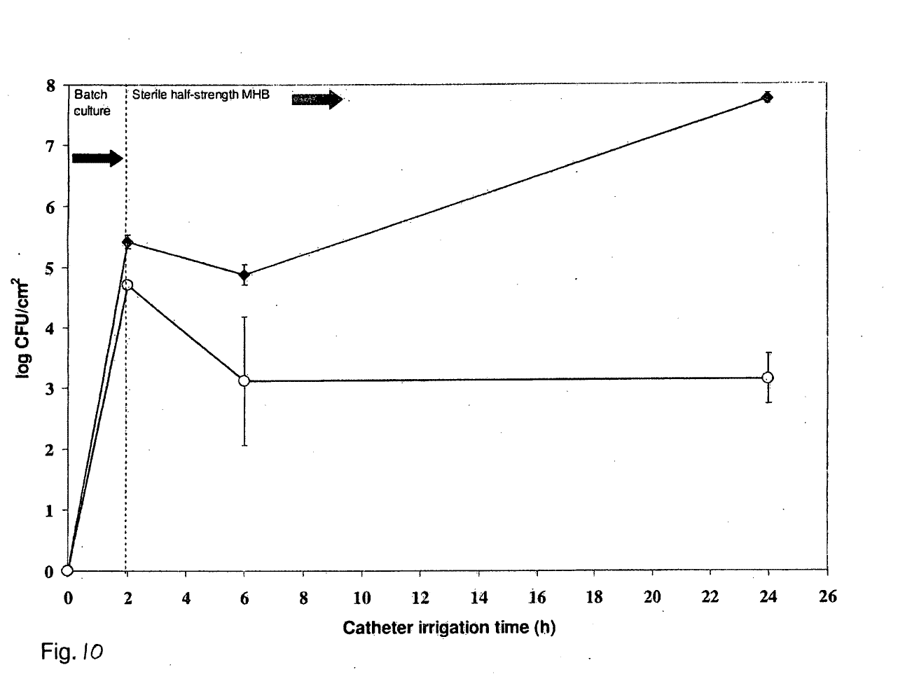

Upon insertion, central venous catheters quickly become coated with platelets, plasma, and host proteins such as albumin, fibrinogen, fibronectin, and laminin. Many authors have shown that the adhesion of staphylococci, including S. epidermidis, to polymer surfaces is not enhanced, and in certain-cases is reduced or impaired, by the presence of such proteins. In this example, catheter surfaces were coated with human serum after phage pre-treatment and prior to biofilm formation. This was carried out mainly to assess what effect the presence of blood proteins would have on phage effectiveness and biofilm formation. A significant increase in the number of viable biofilm cells at 2 h (p=0.0136) and 24 h (p=0.0221) was recorded in the presence of the serum conditioning film (compared with the control). However, it did not effect the ability of the phage to reduce the number of viable cells recovered from the surface (FIG. 10) when compared with non-serum coated catheters pre-treated with phage 456 (in the presence of divalent cations).

### Example 3

### Isolation of Bacteriophage and its Use in Inhibiting a Bacterial Biofilm

This example demonstrates how a bacteriophage specific for a certain species or strain of bacteria can be isolated and used to inhibit bacterial biofilm grown on a surface of an indwelling medical device.

It is well known to those of skill in the art that bacteriophages are present in the excretions of various animals, including livestock (for example, cattle and the like), pets (for example, dogs, cats, birds, and the like), poultry (for example, chickens and the like), and in sewage, and that bacteriophages can be isolated from these sources. Additionally, many of those skilled in the art maintain collections of bacteriophages with known specificities for certain species or strains of bacteria.

In accordance with the present disclosure, any kind of bacteriophages can be employed in order to achieve the objects of the present disclosure, regardless of their source, as long as the bacteriophages have appropriate specificity for target bacteria, for example, Staphylococcus species, such as Staphylococcus aureus, and the coagulase-negative staphylococci (for example, Staphylococcus epidermidis), Pseudomonas species, such as Pseudomonas aeruginosa, bacilli, and enterococci. In other words, there may be employed any bacteriophages that can achieve the objects of the present disclosure by infecting and, in certain embodiments, lysing, specific host bacteria.

For example, members of the MYOVIRIDAE, morphotype A1 family of bacteriophages (such as A, EW, K, Ph5, Ph9, Ph 10, Ph13, P1, P2, P3, P4, P8, P9, P 10, RG, S_{B-1}, (syn=Sb-1), S3K, Twort, φSK311, φ812, 06, 40, 58, 119, 130, 131, 200, and 1623), the SIPHOVIRIDAE, morphotype B2 family of bacteriophages (such as AC3, A8, A10, A13, b594n, D, HK2, N9, N15, P52, P87, S1, S6, Z₄, φRE, 3A, 3B, 3C, 6, 7, 16, 21, 42B, 42C, 42E, 44, 47, 47A, 47C, 51, 54, 54×1, 70, 73, 75, 78, 81, 82, 88, 93, 94, 101, 105, 110, 115, 129/16, 174, 594n, 1363/14, 2460, and NN-Staphylococcus), and the SIPHOVIRIDAE, morphotype B1 family of bacteriophages (such as, AC1, AC2, A6″C″, A9″C″, b⁵⁸¹, CA-1, CA-2, CA-3, CA-4, CA-5, D11, L39×35, L54a, M42, N1, N2, N3, N4, N5, N7, N8, N10, N11, N12, N13, N14, N16, Ph6, Ph12, Ph14, UC-18, U4, U15, S1, S2, S3, S4, S5, X2, Z₁, φB5-2, φD, ω, 11, (syn=φ11), (syn=P11-M15), 15, 28, 28A, 29, 31, 31B, 37, 42D, (syn=P42D), 44A, 48, 51, 52, 52A, (syn=P52A), 52B, 53, 55, 69, 71, (syn=P71), 71A, 72, 75, 76, 77, 79, 80, 80α, 82, 82A, 83A, 84, 85, 86, 88, 88A, 89, 90, 92, 95, 96, 102, 107, 108, 111, 129-26, 130, 130A, 155, 157, 157A, 165, 187, 275, 275A, 275B, 356, 456, 459, 471, 471A, 489, 581, 676, 898, 1139, 1154A, 1259, 1314, 1380, 1405, 1563, 2148, 2638A, 2638B, 2638C, 2731, 2792A, 2792B, 2818, 2835, 2848A, 3619, 5841, and 12100) infect Staphylococcus species (see, for example, The Bacteriophage Ecology Group web site).

Additionally, members of the SIPHOVIRIDAE, morphotype B1 family of bacteriophages (such as af, A7, B3, B33, B39, BI-1, C22, D3, D37, D40, D62, D3112, F7, F10, g, gd, ge, gf, Hw12, Jb19, KF1, L°, OXN-32P, 06N-52P, PCH-1, PC13-1, PC35-1, PH2, PH51, PH93, PH132, PMW, PM13, PM57, PM61, PM62, PM63, PM69, PM105, PM113, PM681, PM682, PO4, PP1, PP4, PP5, PP64, PP65, PP66, PP71, PP86, PP88, PP92, PP401, PP711, PP891, Pssy41, Pssy42, Pssy403, Pssy404, Pssy420, Pssy923, PS4, PS-10, Pz, SD1, SL1, SL3, SL5, SM, φC5, φC11, φC11-1, φC13, φC15, φMO, φX, φ04, φ11, φ240, 2, 2F, 5, 7m, 11, 13, 13/441, 14, 20, 24, 40, 45, 49, 61, 73, 148, 160, 198, 218, 222, 236, 242, 246, 249, 258, 269, 295, 297, 309, 318, 342, 350, 351, 357-1, 400-1, and NN-Pseudomonas), the PODOVIRIDAE, morphotype C1 family of bacteriophages (such as A856, B26, CI-1, CI-2, C5, D, gh-1, F116, HF, H90, K₅, K₆, K104, K109, K166, K267, N₄, N₅, O6N-25P, PE69, Pf, PPN25, PPN35, PPN89, PPN91, PP2, PP3, PP4, PP6, PP7, PP8, PP56, PP87, PP114, PP206, PP207, PP306, PP651, Psp231a, Pssy401, Pssy9220, ps₁, PTB2, PTB20, PTB42, PX1, PX3, PX10, PX12, PX14, PYO70, PYO71, R, SH6, SH133, tf, Ya₅, Ya₇, φBS, ΦKf77, φ-MC, ΦmnF82, φPLS27, φPLS743, φS-1, 1, 2, 2, 3, 4, 5, 6, 7, 7, 8, 9, 10, 11, 12, 12B, 13, 14, 15, 14, 15, 16, 17, 18, 19, 20, 20, 21, 21, 22, 23, 23, 24, 25, 31, 53, 73, 119x, 145, 147, 170, 267, 284, 308, 525, and NN-Pseudomonas), and the MYOVIRIDAE, morphotype A1 family of bacteriophages (such as AI-1, AI-2, B17, B89, CB3, Col 2, Col 11, Col 18, Col 21, C154, C163, C167, C2121, E79, F8, ga, gb, H22, K₁, M4, N₂, Nu, PB-1, (syn=PB 1), pf16, PMN17, PP1, PP8, Psa1, PsP1, PsP2, PsP3, PsP4, PsP5, PS3, PS17, PTB80, PX4, PX7, PYO1, PYO2, PYO5, PYO6, PYO9, PYO10, PYO13, PYO14, PYO16, PYO18, PYO19, PYO20, PYO29, PYO32, PYO33, PYO35, PYO36, PYO37, PYO38, PYO39, PYO41, PYO42, PYO45, PYO47, PYO48, PYO64, PYO69, PYO103, P1K, SLP1, SL2, S₂, UNL-1, wy, Ya₁, Ya₄, Ya₁₁, φBE, φCTX, φC17, φKZ, (syn=ΦKZ), φ-LT, Φmu78, φNZ, φPLS-1, φST-1, φW-14, φ-2, 1/72, 2/79, 3, 3/DO, 4/237, 5/406, 6C, 6/6660, 7, 7v, 7/184, 8/280, 9/95, 10/502, 11/DE, 12/100, 12S, 16, 21, 24, 25F, 27, 31, 44, 68, 71, 95, 109, 188, 337, 352, 1214, and NN-Pseudomonas) infect Pseudomonas species, for example (see, for example, The Bacteriophage Ecology Group web site).

For example, members of the PODOVIRIDAE, morphotype C3 family of bacteriophages (such as C2, C2F, E3, and E62), members of the PODOVIRIDAE, morphotype C1 family of bacteriophages (such as D1, SB24, 2BV, 182, and 225), members of the MYOVIRIDAE, morphotype A1 family of bacteriophages (such as DF₇₈, F1, F2, 1, 2, 4, 14, 41, and 867), and members of the SIPHOVIRIDAE, morphotype B1 family of bacteriophages (such as DS96, H24, M35, P3, P9, SB101, S2, 2BII, 5; 182a, 705, 873, 881, 940, 1051, 1057, 21096C, and NN-Enterococcus) infect enterococcus species (see, for example, The Bacteriophage Ecology Group web site).

Screening of Bacteriophages to determine bacterial specificity, and the ability to selectively lyse pathogenic bacteria, can be carried out by a number of methods well known to those of skill in the art (see, for example, U.S. Pat. No. 6,322,783).

While this disclosure has been described with an emphasis. upon preferred embodiments, it will be obvious to those of ordinary skill in the art that variations of the preferred embodiments may be used and it is intended that the disclosure may be practiced otherwise than as specifically described herein. Accordingly, this disclosure includes all modifications encompassed within the spirit and scope of the disclosure as defined by the claims below.

We claim:

1. An indwelling medical device suitable for introduction into the body of a subject, wherein the device comprises:
one or more surfaces on which a bacterial biofilm can form; and
an effective amount of a bacteriophage composition incorporated into a viscous material carried by one or more of the surfaces, wherein the bacteriophage composition inhibits formation of the bacterial biofilm on the indwelling medical device.

2. The medical device of claim 1, wherein the bacteriophage composition is releasably absorbed by the vicious material.

3. The medical device of claim 1, wherein the viscous material is a gel.

4. The medical device of claim 1, wherein the viscous material comprises a hydrogel.

5. The medical device of claim 1, wherein the bacteriophage composition comprises a plurality of bacteriophages, and the bacteriophage composition inhibits the formation of a polymicrobic biofilm.

6. The medical device of claim 1, wherein the device is made by contacting the bacteriophage composition and a carrier with the one or more surfaces of the device for a sufficient period of time to adhere the effective amount of the bacteriophage to the device, prior to introducing the device into the body.

7. The medical device of claim 1, wherein the bacteriophage composition comprises an effective amount of divalent cations.

8. The medical device of claim 1, wherein the bacteriophage composition comprises an effective amount of Ca²⁺ and Mg²⁺.

9. The medical device of claim 1, wherein the bacteriophage composition comprises a lytic bacteriophage composition.

10. The medical device of claim 1, wherein the bacteriophage composition comprises a staphylococcal bacteriophage composition.

11. The medical device of claim 10, wherein the staphylococcal bacteriophage composition comprises bacteriophage 456.

12. The medical device of claim 10, wherein the staphylococcal bacteriophage composition comprises 10¹⁰ PFU/ml of bacteriophage 456.

13. The medical device of claim 1, wherein the bacteriophage composition comprises a bacteriophage that inhibits the growth of bacteria of the family Enterobacteriacae.

14. The medical device of claim 1, wherein the bacteriophage composition comprises a bacteriophage that inhibits the growth of bacteria of a genus selected from the group consisting of Staphylococcus, Enterococcus, and Streptococcus.

15. The medical device of claim 1, wherein the bacteriophage composition comprises a bacteriophage that inhibits the growth of a bacteria selected from the group consisting of Staphylococcus aureus, Staphylococcus epidermidis, coagulase-negative staphylococci, Pseudomonas aeruginosa, Klebsiella pneumoniae, Escherichia coli, Enterococcusfaecalis, Enterococcusfaecium, Providencia stuarii, Proteus mirabilis, Morganella morganii, Acinetobacter calcoaceticus, Enterobacter aerogenes, Streptococcus agalactiae, Streptococcus avium, Streptococcus bovis, Streptococcus durans, Streptococcus faecalis, Streptococcus pneumoniae, Streptococcus pyogenes, Streptococcus suis, Streptococcus viridans, and Streptococcus salivarius.

16. The medical device of claim 1, wherein the medical device is suitable for surgical implantation within the body.

17. The medical device of claim 1, wherein the bacteriophage composition inhibits biofilm formation for a prolonged time period.

18. The medical device of claim 1, wherein the bacteriophage composition inhibits biofilm formation for at least twenty-four hours.

19. The medical device of claim 1, wherein the device is sterile except for the bacteriophage composition.

20. The medical device of claim 1, wherein the device is a polymeric device.

21. The medical device of claim 1, wherein the device is a catheter, a stent, a shunt, an endotracheal tube, a gastric feeding tube, an artificial joint, an intrauterine device, an artificial voice prosthesis, a needless connector for central venous catheters, a tympanostomy tube, an artificial heart valve, or a pacemaker.

22. The medical device of claim 1, wherein the bacteriophage composition further comprises a surfactant, an antibacterial enzyme, an antibiotic, or combinations thereof.

23. The medical of claim 1, wherein the bacteriophage composition further comprises an antibiotic.

24. The medical device of claim 23, wherein the antibiotic comprises a beta-lactam, a cephalosporin, an aminoglycoside, a sulfonamide, a macrolide, a tetracycline, a silver salt, or combinations thereof.

25. The medical device of claim 1, wherein the device comprises a lumen.

26. The medical device of claim 25, wherein the device is a catheter and wherein the bacteriophage composition is present in the lumen.

27. A method for inhibiting formation of a bacterial biofilm on a surface of an indwelling medical device, comprising contacting the surface of the medical device with an effective amount of a viscous material comprising one or more bacteriophages that inhibit formation of the bacterial biofilm prior to formation of the biofilm.

28. The method of claim 27, wherein the viscous material is a gel.

29. The method of claim 27, wherein the viscous material comprises a hydrogel.

30. The method of claim 27, wherein the one or more bacteriophages are releasably absorbed by the viscous material.

31. The method of claim 27, wherein contacting the surface of the medical device with an effect amount of a viscous material comprising one or more bacteriophages that inhibit formation of the bacterial biofilm prior to formation of the biofilm comprises contacting the surface of the medical device with a viscous material and contacting the viscous material with the one or more bacteriophages such that the one or more bacteriophages are absorbed by the viscous material.

32. The method of claim 31, wherein the viscous material comprises a hydrogel.

33. The method of claim 27, wherein the surface of the medical device is coated with a viscous material and contacting the surface of the medical device with an effective amount of a viscous material comprising one or more bacteriophages that inhibit formation of the bacterial biofilm prior to formation of the biofilm comprises contacting the viscous material with the one or more bacteriophages such that the one or more bacteriophages are absorbed by the viscous material.

34. The method of claim 33, wherein the viscous material comprises a hydrogel.

35. The method of claim 27, wherein the viscous material comprises a plurality of bacteriophages, and the viscous material inhibits the formation of a polymicrobic biofilm.

36. The method of claim 27, wherein the viscous material comprises an effective amount of divalent cations.

37. The method of claim 27, wherein the viscous material comprises an effective amount of Ca²⁺ and Mg²⁺.

38. The method of claim 27, wherein the viscous material inhibits biofilm formation for a prolonged time period.

39. The method of claim 27, wherein the viscous material inhibits biofilm formation for at least twenty-four hours.

40. The method of claim 27, wherein the one or more bacteriophages inhibit formation of a staphylococcal bacteria biofilm.

41. The method of claim 40, wherein the bacterial biofilm comprises S. aureus.

42. The method of claim 40, wherein the bacterial biofilm comprises S. epidermidis.

43. The method of claim 27, wherein the one or more bacteriophages comprise one or more lytic bacteriophages.

44. The method of claim 27, wherein the one or more bacteriophages comprise one or more staphylococcal bacteriophages.

45. The method of claim 27, wherein the viscous material further comprises a surfactant, an antibacterial enzyme, an antibiotic, or combinations thereof.

46. The method of claim 27, wherein the viscous material comprises an antibiotic.

47. The method of claim 46, wherein the antibiotic comprises a beta-lactam, a cephalosporin, an aminoglycoside, a sulfonamide, a macrolide, a tetracycline, a silver salt, or combinations thereof.

48. The method of claim 27, wherein the medical device is suitable for surgical implantation within the body.

49. The method of claim 27, wherein the medical device is a catheter, a stent, a shunt, an endotracheal tube, a gastric feeding tube, an artificial joint, an intrauterine device, an artificial voice prosthesis, a needless connector for central venous catheters, a tympanostomy tube, an artificial heart valve, or a pacemaker.

50. The method of claim 27, wherein the medical device comprises a lumen.

51. The method of claim 50, wherein the medical device is a catheter and wherein the viscous material is present in the lumen.

52. The method of claim 51, wherein the viscous material is present in the lumen of the catheter prior to insertion of the catheter into a subject.

53. The method of claim 51, wherein the viscous material is present in the lumen of the catheter after insertion of the catheter into a subject.

54. The method of claim 27, wherein the one or more bacteriophages comprise a bacteriophage that inhibits the growth of bacteria of the family Enterobacteriacae.

55. The method of claim 27, wherein the one or more bacteriophages comprise a bacteriophage that inhibits the growth of a bacteria of a genus selected from the group consisting of Staphylococcus, Enterococcus, and Streptococcus.

56. The method of claim 27, wherein the one or more bacteriophages comprise a bacteriophage that inhibits the growth of a bacteria selected from the group consisting of Staphylococcus aureus, Staphylococcus epidermidis, coagulase-negative staphylococci, Pseudomonas aeruginosa, Klebsiella pneumonia, Escherichia coli, Enterococcusfaecalis, Enterococcusfaecium, Providencia stuartii, Proteus mirabilis, Morganella morganii, Acinetobacter calcoaceticus, Enterobacter aerogenes, Streptococcus agalactiae, Streptococcus avium, Streptococcus bovis, Streptococcus durans, Streptococcus faecalis, Streptococcus pneumoniae, Streptococcus pyogenes, Streptococcus suis, Streptococcus viridans, and Streptococcus salivarius.

57. A method of making a medical device comprising coating a surface of the medical device with a viscous material and incorporating a bacteriophage into the viscous material before or after coating the surface of the medical device with the viscous material.

58. The method of claim 57, wherein the viscous material is a viscous gel.

59. The method of claim 57, wherein the viscous material comprises a hydrogel.

60. The method of claim 57, wherein the bacteriophage is releasably absorbed by the viscous material.

61. A method of introducing an indwelling medical device into the body of a subject comprising providing a medical device coated with a viscous material comprising a bacteriophage and introducing the medical device into the body of a subject.

62. The method of claim 61, wherein the medical device remains in the body of the subject for at least twenty-four hours.

63. The method of claim 61, wherein the medical device remains in the body of the subject for at least thirty days.

64. The method of claim 61, wherein the medical device remains in the body of the subject for at least a year.

65. The method of claim 61, wherein the bacteriophage is releasably absorbed by the viscous material.

66. The method of claim 61, wherein the viscous material is a gel.

67. The method of claim 61, wherein the viscous material is a hydrogel.
